(19) United States
(12) Patent Application Publication
(10) Pub. No.: US 2004/0076964 A1
(43) Pub. Date: Apr. 22, 2004

(54) Methods for in situ generation of nucleic acid arrays

(76) Inventors: Eric M. Leproust, Campbell, CA (US); Robert E. Woodworth, Sunnyvale, CA (US)

Correspondence Address: AGILENT TECHNOLOGIES, INC., INTELLECTUAL PROPERTY ADMINISTRATION, LEGAL DEPT., P.O. BOX 7599, M/S DL429, LOVELAND, CO, 80537-0599, US

(21) Appl. No.: 10/278,274

(22) Filed: Oct. 22, 2002

(51) Int. Cl. B05D 3/00; C12Q 1/68; C12M 1/34

(52) U.S. Cl. 435/6; 435/287.2; 427/2.11

(57) ABSTRACT

Methods of producing nucleic acid arrays using an in situ nucleic acid synthesis protocol are provided, where the in situ nucleic acid synthesis protocol includes a plurality of cycles, each of which includes: (I) a monomer attachment step; and (II) a functional group, e.g., 5′OH, generation step, the latter of which includes: (a) oxidation and (b) deblocking substeps. A feature of the subject methods is that the deblocking substep is performed by contacting a substrate surface with a deblocking solution made up of a deblocking agent present in an organic solvent, wherein the organic solvent is toluene or another organic solvent having substantially the same vapor pressure. Also provided are the arrays produced using the subject methods, as well as methods for use of the arrays and kits that include the same.

### TECHNICAL FIELD

The field of this invention is nucleic acid arrays.

### BACKGROUND OF THE INVENTION

Arrays of nucleic acids, e.g., “gene chips,” have become an increasingly important tool in the biotechnology industry and related fields. These nucleic acid arrays, in which a plurality of distinct or different nucleic acids are positioned on a solid support surface in the form of an array or pattern, find use in a variety of applications, including gene expression analysis, drug screening, nucleic acid sequencing, mutation analysis, and the like.

A feature of many arrays that have been developed is that each of the distinct nucleic acids of the array is stably attached to a discrete location on the array surface, such that its position remains constant and known throughout the use of the array. Stable attachment is achieved in a number of different ways, including covalent bonding of the polymer to the support surface and non-covalent interaction of the polymer with the surface.

There are two main ways of producing nucleic acid arrays in which the immobilized nucleic acids are covalently attached to the substrate surface, i.e., via in situ synthesis in which the nucleic acid ligand is grown on the surface of the substrate in a step-wise fashion and via deposition of the full ligand, e.g., a presynthesized nucleic acid/polypeptide, cDNA fragment, etc., onto the surface of the array.

Where the in situ synthesis approach is employed, conventional phosphoramidite synthesis protocols are typically used. In phosphoramidite synthesis protocols, the 3′-hydroxyl group of an initial 5′-protected nucleoside is first covalently attached to the polymer support, e.g., a planar substrate surface. Synthesis of the nucleic acid then proceeds by deprotection of the 5′-hydroxyl group of the attached nucleoside, followed by coupling of an incoming nucleoside-3′-phosphoramidite to the deprotected 5′ hydroxyl group (5′-OH). The resulting phosphite triester is finally oxidized to a phosphotriester to complete the internucleotide bond. The steps of deprotection, coupling and oxidation are repeated until a nucleic acid of the desired length and sequence is obtained.

Optionally, a capping reaction may be used after the coupling and/or after the oxidation to inactivate the growing DNA chains that failed in the previous coupling step, thereby avoiding the synthesis of inaccurate sequences.

The chemical group conventionally used for the protection of nucleoside 5′-hydroxyls is dimethoxytrityl (“DMT”), which group is removable with acid. This acid-labile protecting group provides a number of advantages for working with both nucleosides and oligonucleotides. For example, the DMT group can be introduced onto a nucleoside regioselectively and in high yield. Also, the lipophilicity of the DMT group greatly increases the solubility of nucleosides in organic solvents, and the carbocation resulting from acidic deprotection gives a strong chromophore, which can be used to indirectly monitor coupling efficiency. In addition, the hydrophobicity of the group can be used to aid separation on reverse-phase HPLC.

However, use of DMT as a hydroxyl-protecting group in nucleic acid synthesis is also problematic. The N-glycosidic linkages of nucleic acids are susceptible to acid catalyzed cleavage, and even when the protocol is optimized, recurrent removal of the DMT group with acid during synthesis results in depurination. The N-6-benzoyl-protected deoxyadenosine nucleotide is especially susceptible to glycosidic cleavage, resulting in a substantially reduced yield of the final nucleic acid. In the context of in situ nucleic acid array synthesis, glycisidic bond cleavage as described above leads to cleavage of the phosphotriester during deprotection, which results in the production of shorter sequences and inaccurate signal intensities.

In the synthesis of nucleic acids on the surface of a nucleic acid array or “gene chip,” reactive deoxynucleoside phosphoramidites are successively applied, in molecular amounts exceeding the molecular amounts of target hydroxyl groups of the substrate or growing oligonucleotide polymers, to specific cells of the high-density array, where they chemically bond to the target hydroxyl groups. Then, unreacted deoxynucleoside phosphoramidites from multiple cells of the high-density array are washed away, oxidation of the phosphite bonds joining the newly added deoxynucleosides to the growing oligonucleotide polymers to form phosphate bonds is carried out, and unreacted hydroxyl groups of the substrate or growing oligonucleotide polymers are chemically capped to prevent them from reacting with subsequently applied deoxynucleoside phosphoramidites. Optionally, the capping reaction may be done prior to oxidation.

While nucleic acid arrays have been manufactured using in situ synthesis techniques, as described above, the problems associated with the use of DMT are exacerbated in protocols where “microscale” parallel reactions are taking place on a very dense, packed surface, e.g., as occurs in the fabrication of many types of nucleic acid arrays. Applications in the field of genomics and high throughput screening have fueled the demand for precise chemistry in such a context. Thus, increasingly stringent demands are placed on the chemical synthesis cycle as it was originally conceived, and the problems associated with conventional methods for synthesizing oligonucleotides are rising to unacceptable levels in these expanded applications.

Accordingly, there is continued interest in the development of new protocols for producing: nucleic acid arrays via in situ synthesis. Of particular interest would be the development of a protocol that was amenable to automated applications and resulted in high quality arrays by at least reducing, if not substantially eliminating, depurination side reactions during deblocking.

Relevant Literature

U.S. Patents of interest include: U.S. Pat. Nos. 6,020,475; 6,222,030; and 6,300,137.

### SUMMARY OF THE INVENTION

Methods of producing nucleic acid arrays using an in situ nucleic acid synthesis protocol are provided, where the in situ nucleic acid synthesis protocol includes a plurality of cycles, each of which includes: (I) a monomer attachment step; and (II) an internucleotide linkage stabilization and 5′ functional group, e.g., 5′OH, generation step, the latter of which includes: (a) oxidation and (b) deblocking substeps, and optionally a capping substep. A feature of the subject methods is that the deblocking substep is performed by contacting a substrate surface with a deblocking solution made up of an acid deblocking agent present in an organic solvent, wherein the organic solvent is toluene or another solvent that has a low vapor pressure. Also provided are the arrays produced using the subject methods, as well as methods for use of the arrays and kits that include the same.

### BRIEF DESCRIPTION OF THE FIGURES

FIG. 1 provides a schematic of an automated array processing device in which the subject methods may be employed.

FIG. 2 provides a flow diagram of representative array fabrication protocol according to the subject methods.

### DEFINITIONS

The term “nucleic acid” as used herein means a polymer composed of nucleotides, e.g. deoxyribonucleotides or ribonucleotides, or compounds produced synthetically (e.g. PNA as described in U.S. Pat. No. 5,948,902 and the references cited therein) which can hybridize with naturally occurring nucleic acids in a sequence specific manner analogous to that of two naturally occurring nucleic acids, e.g., can participate in hybridization reactions, i.e., cooperative interactions through Pi electrons stacking and hydrogen bonds, such as Watson-Crick base pairing interactions, Wobble interactions, etc.

The terms “ribonucleic acid” and “RNA” as used herein mean a polymer composed of; ribonucleotides.

The terms “deoxyribonucleic acid” and “DNA” as used herein mean a polymer composed of deoxyribonucleotides.

The term “oligonucleotide” as used herein denotes single stranded nucleotide multimers of from about 10 to 100 nucleotides and up to 200 nucleotides in length.

The term “polynucleotide” as used herein refers to single or double stranded polymer composed of nucleotide monomers of generally greater than 100 nucleotides in length.

The term “monomer” as used herein refers to a chemical entity that can be covalently linked to one or more other such entities to form an polymer. Of particular interest to the present application are nucleotide “monomers” that have first and second sites (e.g., 5′ and 3′ sites) suitable for binding to other like monomers by means of standard chemical reactions (e.g., nucleophilic substitution), and a diverse element which distinguishes a particular monomer from a different monomer of the same type (e.g., a nucleotide base, etc.). In the art synthesis of nucleic acids of this type utilizes an initial substrate-bound monomer that is generally used as a building-block in a multi-step synthesis procedure to form a complete nucleic acid.

The term “oligomer” is used herein to indicate a chemical entity that contains a plurality of monomers. As used herein, the terms “oligomer” and “polymer” are used interchangeably, as it is generally, although not necessarily, smaller “polymers” that are prepared using the functionalized substrates of the invention, particularly in conjunction with combinatorial chemistry techniques. Examples of oligomers and polymers include polydeoxyribonucleotides (DNA), polyribonucleotides (RNA), other polynucleotides which are C-glycosides of a purine or pyrimidine base. In the practice of the instant invention, oligomers will generally comprise about 2-60 monomers, preferably about 10-60, more preferably about 50-60 monomers.

The term “sample” as used herein relates to a material or mixture of materials, typically, although not necessarily, in fluid form, containing one or more components of interest.

The terms “nucleoside” and “nucleotide” are intended to include those moieties which contain not only the known purine and pyrimidine bases, but also other heterocyclic bases that have been modified. Such modifications include methylated purines or pyrimidines, acylated purines or pyrimidines, alkylated riboses or other heterocycles. In addition, the terms “nucleoside” and “nucleotide” include those moieties that contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, or are functionalized as ethers, amines, or the like.

The terms “protection” and “deprotection” as used herein relate, respectively, to the addition and removal of chemical protecting groups using conventional materials and techniques within the skill of the art and/or described in the pertinent literature; for example, reference may be had to Greene et al., Protective Groups in Organic Synthesis, 2nd Ed., New York: John Wiley & Sons, 1991. Protecting groups prevent the site to which they are attached from participating in the chemical reaction to be carried out.

“Optional” or “optionally” means that the subsequently described circumstance may or may not occur, so that the description includes instances where the circumstance occurs and instances where it does not. For example, the phrase “optionally substituted” means that a non-hydrogen substituent may or may not be present, and, thus, the description includes structures wherein a non-hydrogen substituent is present and structures wherein a non-hydrogen substituent is not present.

An “array,” includes any two-dimensional or substantially two-dimensional (as well as a three-dimensional) arrangement of addressable regions bearing a particular chemical moiety or moieties (e.g., biopolymers such as polynucleotide or oligonucleotide sequences (nucleic acids), polypeptides (e.g., proteins), carbohydrates, lipids, etc.) associated with that region. In the broadest sense, the preferred arrays are arrays of polymeric binding agents, where the polymeric binding agents may be any of: polypeptides, proteins, nucleic acids, polysaccharides, synthetic mimetics of such biopolymeric binding agents, etc. In many embodiments of interest, the arrays are arrays of nucleic acids, including oligonucleotides, polynucleotides, cDNAs, mRNAs, synthetic mimetics thereof, and the like. Where the arrays are arrays of nucleic acids, the nucleic acids may be covalently attached to the arrays at any point along the nucleic acid chain, but are generally attached at one of their termini (e.g. the 3′ or 5′ terminus). Sometimes, the arrays are arrays of polypeptides, e.g., proteins or fragments thereof.

Any given substrate may carry one, two, four or more or more arrays disposed on a front surface of the substrate. Depending upon the use, any or all of the arrays may be the same or different from one another and each may contain multiple spots or features. A typical array may contain more than ten, more than one hundred, more than one thousand more ten thousand features, or even more than one hundred thousand features, in an area of less than 20 cm² or even less than 10 cm². For example, features may have widths (that is, diameter, for a round spot) in the range from a 10 μm to 1.0 cm. In other embodiments each feature may have a width in the range of 1.0 μm to 1.0 mm, usually 5.0 μm to 500 μm, and more usually 10 μm to 200 μm. Non-round features may have area ranges equivalent to that of circular features with the foregoing width (diameter) ranges. At least some, or all, of the features are of different compositions (for example, when any repeats of each feature composition are excluded the remaining features may account for at least 5%, 10%, or 20% of the total number of features). Interfeature areas will typically (but not essentially) be present which do not carry any polynucleotide (or other biopolymer or chemical moiety of a type of which the features are composed). Such interfeature areas typically will be present where the arrays are formed by processes involving drop deposition of reagents but may not be present when, for example, photolithographic array fabrication processes are used. It will be appreciated though, that the interfeature areas, when present, could be of various sizes and configurations.

Each array may cover an area of less than 100 cm², or even less than 50 cm², 10 cm² or 1 cm². In many embodiments, the substrate carrying the one or more arrays will be shaped generally as a rectangular solid (although other shapes are possible), having a length of more than 4 mm and less than 1 m, usually more than 4 mm and less than 600 mm, more usually less than 400 mm; a width of more than 4 mm and less than 1 m, usually less than 500 mm and more usually less than 400 mm; and a thickness of more than 0.01 mm and less than 5.0 mm, usually more than 0.1 mm and less than 2 mm and more usually more than 0.2 and less than 1 mm. With arrays that are read by detecting fluorescence, the substrate may be of a material that emits low fluorescence upon illumination with the excitation light. Additionally in this situation, the substrate may be relatively transparent to reduce the absorption of the incident illuminating laser light and subsequent heating if the focused laser beam travels too slowly over a region. For example, substrate 10 may transmit at least 20%, or 50% (or even at least 70%, 90%, or 95%), of the illuminating light incident on the front as may be measured across the entire integrated spectrum of such illuminating light or alternatively at 532 nm or 633 nm.

Arrays can be fabricated using drop deposition from pulse jets of either polynucleotide precursor units (such as monomers) in the case of in situ fabrication, or the previously obtained polynucleotide. Such methods are described in detail in, for example, the previously cited references including U.S. Pat. No. 6,242,266, U.S. Pat. No. 6,232,072, U.S. Pat. No. 6,180,351, U.S. Pat. No. 6,171,797, U.S. Pat. No. 6,323,043, U.S. patent application Ser. No. 09/302,898 filed Apr. 30, 1999 by Caren et al., and the references cited therein. As already mentioned, these references are incorporated herein by reference. Other drop deposition methods can be used for fabrication, as previously described herein. Also, instead of drop deposition methods, photolithographic array fabrication methods may be used such as described in U.S. Pat. No. 5,599,695, U.S. Pat. No. 5,753,788, and U.S. Pat. No. 6,329,143. Interfeature areas need not be present particularly when the arrays are made by photolithographic methods as described in those patents.

An array is “addressable” when it has multiple regions of different moieties (e.g., different polynucleotide sequences) such that a region (i.e., a “feature” or “spot” of the array) at a particular predetermined location (i.e., an “address”) on the array will detect a particular target or class of targets (although a feature may incidentally detect non-targets of that feature). Array features are typically, but need not be, separated by intervening spaces. In the case of an array, the “target” will be referenced as a moiety in a mobile phase (typically fluid), to be detected by probes (“target probes”) which are bound to the substrate at the various regions. However, either of the “target” or “target probe” may be the one which is to be evaluated by the other (thus, either one could be an unknown mixture of polynucleotides to be evaluated by binding with the other). A “scan region” refers to a contiguous (preferably, rectangular) area in which the array spots or features of interest, as defined above, are found. The scan region is that portion of the total area illuminated from which the resulting fluorescence is detected and recorded. For the purposes of this invention, the scan region includes the entire area of the slide scanned in each pass of the lens, between the first feature of interest, and the last feature of interest, even if there exist intervening areas which lack features of interest. An “array layout” refers to one or more characteristics of the features, such as feature positioning on the substrate, one or more feature dimensions, and an indication of a moiety at a given location. “Hybridizing” and “binding”, with respect to polynucleotides, are used interchangeably.

By “remote location,” it is meant a location other than the location at which the array is present and hybridization occurs. For example, a remote location could be another location (e.g., office, lab, etc.) in the same city, another location in a different city, another location in a different state, another location in a different country, etc. As such, when one item is indicated as being “remote” from another, what is meant is that the two items are at least in different rooms or different buildings, and may be at least one mile, ten miles, or at least one hundred miles apart. “Communicating” information references transmitting the data representing that information as electrical signals over a suitable communication channel (e.g., a private or public network). “Forwarding” an item refers to any means of getting that item from one location to the next, whether by physically transporting that item or otherwise (where that is possible) and includes, at least in the case of data, physically transporting a medium carrying the data or communicating the data. An array “package” may be the array plus only a substrate on which the array is deposited, although the package may include other features (such as a housing with a chamber). A “chamber” references an enclosed volume (although a chamber may be accessible through one or more ports). It will also be appreciated that throughout the present application, that words such as “top,” “upper,” and “lower” are used in a relative sense only.

### DESCRIPTION OF THE SPECIFIC EMBODIMENTS

Methods of producing nucleic acid arrays using an in situ nucleic acid synthesis protocol are provided, where the in situ nucleic acid synthesis protocol includes a plurality of cycles, each of which includes: (I) a monomer attachment step; and (II) an internucleotide linkage stabilization and a 5′ functional group, e.g., 5′OH, generation step, the latter of which includes: (a) oxidation and (b) deblocking substeps, where an optional capping substep may also be employed. A feature of the subject methods is that the deblocking substep is performed by contacting a substrate surface with a deblocking solution made up of an acid deblocking agent present in an organic solvent, wherein the organic solvent is toluene or another organic solvent having a low vapor pressure, e.g., one that is substantially the same as the vapor pressure of toluene. Also provided are the arrays produced using the subject methods, as well as methods for use of the arrays and kits that include the same.

Before the subject invention is described further, it is to be understood that the invention is not limited to the particular embodiments of the invention described below; as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

In this specification and the appended claims, the singular forms “a,” “an” and “the” include plural reference unless the context clearly dictates otherwise. Unless defined otherwise, all technical and scientific terms used herein have the same meaning as commonly understood to one of ordinary skill in the art to which this invention belongs.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

All publications mentioned herein are incorporated herein by reference for the purpose of describing and disclosing the invention components which are described in the publications which might be used in connection with the presently described invention.

As summarized above, the subject invention provides methods of producing nucleic acid arrays, as well as the arrays produced thereby and various applications of using the same. In further describing the subject invention, the subject methods are described first in greater detail, followed by a more in-depth review of the arrays produced by the subject methods, representative applications in which the arrays find use, and kits for use in practicing the subject methods.

Methods

As summarized above, the subject invention provides methods of producing nucleic acid arrays. More specifically, the subject invention provides methods of producing nucleic acid arrays by in situ synthesis of two or more distinct nucleic acids on the surface of a solid support. The in situ synthesis protocol employed in the subject invention can be viewed as an iterative process that includes two or more cycles, where each cycle includes of the following steps: (I) a monomer attachment step in which a blocked nucleoside monomer is covalently bonded to two or more distinct locations, e.g., at least a first and second location, of a functional group, e.g., hydroxyl, amino, etc., displaying surface of a solid support; and (II) an internucleotide linkage stabilization and 5′ functional group generation step in which the phosphite triester linkage is oxidized and functional groups are generated at the blocked ends of the resultant attached blocked nucleotides by removal of the blocking groups for addition of subsequent nucleoside monomers.

In certain embodiments of interest, each cycle includes of the following steps: (I) a monomer attachment step in which a 5′OH blocked nucleoside monomer is covalently bonded to two or more distinct locations, e.g., at least a first and second location, of a hydroxyl functional group displaying surface of a solid support, e.g., a nascent planar surface of a solid support displaying hydroxyl functional groups or a surface displaying intermediate nucleic acids having 5′OH groups; and (II) an internucleotide linkage stabilization and 5′OH generation step in which the phosphite triester linkage is oxidized and hydroxyl groups are generated at the 5′ ends of the resultant attached blocked nucleotides by removal of the blocking groups for addition of subsequent nucleoside monomers, where this step includes oxidizing and deblocking substeps, as well as optionally a capping substep. Each of these cycle steps is now described separately in greater detail in terms of these particular embodiments. However, the scope of the invention is not so limited—the invention being described in terms of these particular representative embodiments for ease of description only.

Monomer Attachment Step

In the monomer attachment step of each cycle, one or more different 5′OH blocked nucleoside monomers is contacted with one or more different locations of a substrate surface that displays hydroxyl functional groups, such that the nucleoside monomers become covalently bound to the surface, e.g., via a nucleophilic substitution reaction between the an activated (e.g., protonated) phosphoramidite moiety of the blocked nucleoside monomer and the surface displayed hydroxyl functionality. The surface displayed hydroxyl functionality may be on the surface of a nascent substrate, or may be at the 5′ end of a growing nucleic acid, depending on the particular point in the synthesis protocol. For example, at the beginning of a particular synthesis protocol, the surface displayed hydroxyl functional groups are immediately on the surface of a solid support or substrate. In contrast, following one or more cycles of a given synthesis protocol, the surface displayed functional groups are present at the 5′ ends of growing nucleic acids which, in turn, are covalently bonded to the surface of the solid support.

As such, at the beginning of any array synthesis protocol, the first step is to provide a substrate having a surface that displays hydroxyl functional groups, where the hydroxyl functional groups are employed in the covalent attachment of the growing nucleic acid ligands to the substrate surface during synthesis. The substrate may be any convenient substrate that finds use in biopolymeric arrays. In general, the substrate may be rigid or flexible. By rigid is meant that the support is solid and does not readily bend, i.e., the support is not flexible. As such, rigid substrates are sufficient to provide physical support and structure to the nucleic acid spots present thereon. Furthermore, when the rigid supports of the subject invention are bent, they are prone to breakage. The substrates may be fabricated from a variety of materials. In certain embodiments, e.g., where one is interested in the production of nucleic acid arrays for use in research and related applications, the materials from which the substrate may be fabricated should ideally exhibit a low level of non-specific binding during hybridization events. In many situations, it will also be preferable to employ a material that is transparent to visible and/or UV light. For rigid substrates, specific materials of interest include: silicon; glass; plastics; e.g., polytetrafluoroethylene, polypropylene, polystyrene, polycarbonate, and blends thereof, and the like; metals, e.g. gold, platinum, and the like; etc.: The surface may be modified with one or more different layers of compounds that serve to modify the properties of the surface in a desirable manner. Such modification layers, when present, will generally range in thickness from a monomolecular thickness to about 1 mm, usually from a monomolecular thickness to about 0.1 mm and more usually from a monomolecular thickness to about 0.001 mm. Modification layers of interest include: inorganic and organic layers such as metals, metal oxides, conformal silica or glass coatings, polymers, small organic molecules and the like. Polymeric layers of interest include layers of: peptides, proteins, polynucleic acids or mimetics thereof, e.g. peptide nucleic acids and the like; polysaccharides, phospholipids, polyurethanes, polyesters, polycarbonates, polyureas, polyamides, polyethyleneamines, polyarylene sulfides, polysiloxanes, polyimides, polyacetates, and the like, where the polymers may be hetero- or homopolymeric, and may or may not have separate functional moieties attached thereto, e.g. conjugated. The particular surface chemistry will be dictated by the specific process to be used in polymer synthesis, as described in greater detail infra. However, as mentioned above, the substrate that is initially employed has a surface that displays hydroxyl functional groups.

As mentioned above, nucleic acid arrays are produced according to the subject invention by synthesizing nucleic acid polymers using conventional phosphoramidite solid phase nucleic acid synthesis chemistry where the solid support is a substrate as described above. Phosphoramidite based chemical synthesis of nucleic acids is well known to those of skill in the art, being reviewed above and in U.S. Pat. No. 4,415,732, the disclosure of the latter being herein incorporated by reference.

To produce nucleic acid arrays according to the subject methods, a substrate surface as described above having the appropriate surface groups, e.g. —OH groups, present on its surface, is obtained. Since the synthesis protocol must be carried out under anhydrous conditions, all reactions are carried out in a non-aqueous, typically organic solvent layer on the substrate surface, where the solvent layer is acetonitrile in many embodiments.

Next, the first residues of each nucleic acid to be synthesized on the array are covalently attached to the substrate surface via reaction with the surface bound —OH groups. Depending on whether the first nucleotide residue of each nucleic acid to be synthesized on the array is the same or different, different protocols for this step may be followed. Where each of the nucleic acids to be synthesized on the substrate surface have the same initial nucleotide at the 3′ end, the entire surface of the substrate is contacted with the blocked, activated nucleoside under conditions sufficient for coupling of the activated nucleoside to the reactive groups, e.g. —OH groups, present on the substrate surface to occur. In these embodiments, the entire surface of the array may be contacted with the fluid composition containing the activated nucleoside using any convenient protocol, such as flooding the surface of the substrate with the activated nucleoside solution, immersing the substrate in the solution of activated nucleoside, etc. The fluid composition is typically a fluid composition of the blocked nucleoside in an organic solvent, e.g., acetonitrile, where the fluid composition typically includes an activating agent, e.g., tetrazole, benzoimidazolium triflate (“BZT”), S-ethyl tetrazole, and dicyanoimidazole, etc.

Alternatively, where the initial residue of the various nucleic acids differs among the nucleic acids, one or more sites on the substrate surface are individually contacted with fluid compositions of the appropriate blocked, activated nucleoside. In this latter embodiment, any convenient protocol for selectively contacting a particular site, region or cell of a substrate surface with a fluid composition of the activated nucleoside may be employed. Of particular interest in many embodiments is the use of pulse-jet deposition protocols, such as those described in U.S. Pat. No. 6,300,137; the disclosure of which is herein incorporated by reference. In these embodiments, two or more different fluid compositions of activated, blocked nucleosides, which fluid compositions differ from each other in terms of the activated nucleoside present therein, are each pulse-jetted onto one or more distinct locations of the surface, where the locations are dictated by the sequence of the desired nucleic acid at each location.

The activated nucleoside monomers employed in this attachment step of each cycle of the subject synthesis methods are blocked at their 5′-OH functionalities (ends) with an acid labile blocking group. By acid labile blocking group is meant that the group is cleaved in the presence of an acid to yield a 5′-OH functionality. In many embodiments, the acid labile blocking group is DMT, as described above.

The above step of the subject protocols results in a “blocked monomer attached substrate” where the surface is characterized by the presence of DMT blocked nucleoside monomers covalently attached to the surface of a solid support, either directly if the blocked monomers are the first residues of to be synthesized surface bound nucleic acid ligands, or through a growing nucleic acid ligands, i.e., where blocked monomers are at the end of growing nucleic acid chains. This resultant “blocked monomer attached substrate” is then subjected to the next step of the subject synthesis cycle, i.e., the 5′OH generation step.

Generation of 5′OH Hydroxyl Functionalities

As summarized above, following covalent attachment of activated, blocked nucleoside monomers to one or more locations of the substrate surface, 5′OH hydroxyl moieties are then generated on the surface so that the synthesis cycle can be repeated with a new round of activated, blocked nucleoside monomers. This generation step typically includes the following substeps: (a) oxidation; (b) capping; and (c) deblocking.

A feature of the subject methods is that each of these steps is accomplished by contacting the entire surface of the substrate with an appropriate solution, i.e., an oxidation solution, a capping solution or a deblocking solution, where excess solution employed in a given substep is removed from the surface prior to performing the next substep. Contact of the entire surface may be achieved using any convenient technique, e.g., flooding the surface with the appropriate solution, immersing the substrate in a volume of the appropriate solution, etc., where in many embodiments, the entire substrate is immersed in a volume of the appropriate solution, e.g., in a tank or analogous container element that includes a volume of the appropriate solution. In other embodiments, the surface is coated by a thin layer of solution, e.g. by using spray coating

In many embodiments, performance of each substep includes immersing the substrate in an adequate volume of the appropriate solution so that the entire surface of the substrate is contacted with the solution. For example, the substrate can be placed in a tank that includes a sufficient volume of the appropriate solution so that the substrate can be immersed in the solution.

Following contact with the appropriate solution, excess solution is removed from the surface before contact with the next solution of the next substep. Excess fluid is removed using any convenient protocol, including evaporation, dripping etc., where in many embodiments a dripping protocol is employed, in which the substrate is removed from the volume of solution and held over the volume of solution such that excess solution drips off of the substrate surface and back into the volume of solution.

In performing the above-described substeps, while the order of oxidation and blocking may be reversed, the deblocking step is typically performed following capping/oxidation. As such, the capping/oxidation steps are described together first, followed by a description of the deblocking step. It should be noted that capping before oxidation also prevents formation of branched DNA, while capping after oxidation also removes moisture introduced by the oxidation. In some protocols, capping is done before and after oxidation. As such, capping may be performed before oxidation, after oxidation, or both before and after oxidation.

Oxidation

Oxidation results in the conversion of phosphite triesters present on the substrate surface following coupling to phosphotriesters. Oxidation is accomplished by contacting the surface with an oxidizing solution, as described above, which solution includes a suitable oxidating agent, such as I₂/H₂O/Pyridine/THF. Following contact of the surface with the oxidizing solution, excess is removed as described above.

Optional Capping

In addition, unreacted hydroxyl groups may be (though not necessarily) capped, e.g., using any convenient capping agent, as is known in the art. This optional capping is accomplished by contacting the surface with an capping solution, as described above, which solution includes a suitable capping agent, such as a solution of acetic anhydride, pyridine or 2,6-lutidine (2,6-dimethylpyridine), and tetrahydrofuran (“THF”); a solution of 1-methyl-imidazole in THF; etc. Following contact of the surface with the oxidizing solution, excess oxidizing solution is removed as described above.

Deblocking

The next substep in the subject methods is the deblocking step, where acid labile protecting groups present at the 5′ ends of the growing nucleic acid molecules on the substrate are removed to provide free 5′ OH moieties, e.g., for attachment of subsequent monomers, etc. In this deblocking step, the entire substrate surface is contacted with a deblocking or deprotecting agent as described above. The substrate surface is incubated for a sufficient period of time under appropriate conditions for all available protecting groups to be cleaved from the nucleotides that they are protecting.

A feature of the subject methods is that the deblocking solution includes an acid present in an organic solvent that has a sufficiently lowvapor pressure such that, under the synthesis conditions employed, depurination reactions resulting from the increase in effective acid deblocking agent during evaporation of the solvent from the surface do not occur to any significant extent. The vapor pressure of the organic solvent that is employed in the deblocking solution is typically at least substantially the same as toluene, by which is meant that the vapor pressure is not more than about 350% and usually not more than about 150% of the vapor pressure of toluene at a given set of temperature/pressure conditions. In certain embodiments, the organic solvent is one that has a vapor pressure that is less than about 13 KPa, usually less than about 8 KPa and more usually less than about 5 KPa at standard temperature and pressure conditions i.e., STP conditions (0° C.; 1 ATM). A variety of organic solvents are of interest, where such solvents include, but are not limited to: toluene, xylene (o, m, p), ethylbenzene, perfluoro-n-heptane, perfluoro decalin, chlorobenzene, 1,2 dichloroethane, 1,1,2 trichloroethane, 1,1,2,2 tetrachloroethane, pentachloroethane, and the like; where in many embodiments, the organic solvent that is employed is toluene. The acid deblocking agent employed in the deblocking solution may vary, where representative acids of interest include, but are not limited to: acetic acids, e.g., acetic acid, mono acetic acid, dichloroacetic acid, trichloroacetic acid, monofluoroacetic acid, difluoroacetic acid, trifluoroacetic acid, and the like. The amount of acid in the solution is sufficient to remove blocking groups, and typically ranges between about 0.1 and 20%, more typically ranges between 1 and 3%, as is known in the art.

It must be noted that the evaporation-induced depurination described in the previous paragraph increases as function of the length of the DNA attached on the surface. First, as the DNA length increases, the number of potential sites increases, thus the depurination frequency. Second, as the DNA length increases, the surface energy difference between the features and the unreacted surface increases. In other words, the feature areas become increasingly hydrophilic while the hydrophilicity/hydrophobicity of unreacted area is unchanged. As a result, as the DNA length increases, an increase is observed in the volume of the droplet present on each feature when the support immersion is discontinued. Therefore the amount of deblocking agent available for depurination increases, as well as its effective concentration during solvent evaporation.

Contact of the substrate surface with a deblocking solution results in removal of the protecting groups from the blocked substrate bound residues. As such, this step results in the deprotection of the nucleotide residues on the substrate surface. Following deprotection, the deblocking solution is removed from the surface of the substrate, where removal of the deblocking solution can be accomplished using any convenient protocol, as described above.

As indicated above, in many embodiments a dripping protocol is employed to remove the deblocking solution. In certain of these embodiments, the time length for the dripping step is selected so as to minimize evaporation of the organic solvent of the deblocking solution. In these embodiments, the dripping time typically ranges from about 1 sec to about 60 sec, usually from about 1 s to about 20 sec and more usually from about 5 s to about 10 sec.

In other embodiments, impinging gas jets can be employed to quickly remove excess solution from the substrate surface after emersion. It is also common to use fluid surface tension to remove excess solution from the substrate. As the substrate is pulled from a still surface emersion vessel, fluid surface tension wicks away excess solution from the substrate at the interface. However, due to the increasingly hydrophilic surface of the DNA features, as more layers are built, more solution can be dragged out on the spots.

In certain embodiments, a wash step is performed prior to the deblocking substep. In these embodiments, the surface of the substrate is washed with an excess of the same organic solvent that is employed in the deblocking step.

Removal of the deblocking agent results in a substrate surface in which the nucleotide residues are deprotected. In others words, removal of the deblocking agent results in the production of an array of nucleotide residues stably associated with the substrate surface, where the nucleotide residues on the array surface have 5′-OH groups available for reaction with an activated nucleotide in subsequent cycles.

The above steps of: (a) monomer attachment; and (b) 5′OH hydroxyl regeneration are repeated a number of times with additional nucleotides until each of the desired nucleic acids on the substrate surface are produced. By choosing which sites are contacted with which activated nucleotides, e.g. A, G, C &T, an array having polymers of desired sequence and spatial location is readily achieved.

As such, the above cycles of monomer attachment and hydroxyl moiety regeneration result in the production of an array of desired nucleic acids. The resultant nucleic acid arrays can be employed in a variety of different applications, as described in greater detail below.

The above method steps may be carried out manually or with a suitable automated device, where in many embodiments a suitable automated device is employed. Of particular interest is an automated device that can automatically transfer a substrate from an activated monomer deposition location, i.e., a “writer station” to a surface processing station where the above steps of capping, oxidation and deblocking are carried out, e.g., a wet chemical processing station in which the substrate is automatically transferred from one tank to another in a sequential fashion. A representative automated manufacturing device that is adapted to perform the subject methods is described in Example 1.

As indicated above, the above description describing use of 5′OH functional groups, acid labile blocking groups, such as DMT and the use of an acid deblocking agent, are merely representative. Various modifications may be made and still fall within the scope of the invention. For example, other functional groups may be employed, e.g., amine functional groups. In yet other embodiments, base labile blocking groups may be employed, where such groups and the use thereof are described in U.S. Pat. No. 6,222,030; the dislcosure of which is herein incorporated by reference. In these latter types of embodiments, the acid deblocking agent described above is replaced with a base deblocking agent.

Arrays

Also provided by the subject invention are arrays of nucleic acids produced according to the subject methods, as described above. The subject arrays include at least two distinct nucleic acids that differ by monomeric sequence immobilized on e.g., covalently to, different and known locations on the substrate surface. In certain embodiments, each distinct nucleic acid sequence of the array is typically present as a composition of multiple copies of the polymer on the substrate surface, e.g. as a spot on the surface of the substrate. The number of distinct nucleic acid sequences, and hence spots or similar structures, present on the array may vary, but is generally at least, usually at least 5 and more usually at least 10, where the number of different spots on the array may be as a high as 50, 100, 500, 1000, 10,000 or higher, depending on the intended use of the array. The spots of distinct polymers present on the array surface are generally present as a pattern, where the pattern may be in the form of organized rows and columns of spots, e.g. a grid of spots, across the substrate surface, a series of curvilinear rows across the substrate surface, e.g. a series of concentric circles or semi-circles of spots, and the like. The density of spots present on the array surface may vary, but will generally be at least about 10 and usually at least about 100 spots/cm², where the density may be as high as 10⁶ or higher, but will generally not exceed about 10⁵ spots/cm². In other embodiments, the polymeric sequences are not arranged in the form of distinct spots, but may be positioned on the surface such that there is substantially no space separating one polymer sequence/feature from another.

As indicated above, the arrays are arrays of nucleic acids, including oligonucleotides, polynucleotides, cDNAs, mRNAs, synthetic mimetics thereof, and the like. Where the arrays are arrays of nucleic acids, the nucleic acids may be covalently attached to the arrays at any point along the nucleic acid chain, but are generally attached at one of their termini, e.g. the 3′ or 5′ terminus. In other embodiments, the arrays are arrays of polypeptides, e.g., proteins or fragments thereof.

A feature of the subject arrays, which feature results from the protocol employed to manufacture the arrays, is that each probe location of the arrays is highly uniform in terms of probe composition, since substantially no depurination side reactions occur during the array processing, if any. As such, the proportion of full-length sequence within each feature is higher as compared to arrays produced using analogous protocols but not with toluene or an analogous solvent, as described herein (e.g., at least about 5-fold higher, often at least about 10-fold higher, such as at least about 25-, 50- or 75-fold higher), and the length distribution within each feature is less skewed towards shorter sequences. As a result, background noise and non selective signal are reduced in the hybridization signal.

Utility

The subject arrays find use in a variety of different applications, where such applications are generally analyte detection applications in which the presence of a particular analyte in a given sample is detected at least qualitatively, if not quantitatively. Protocols for carrying out such assays are well known to those of skill in the art and need not be described in great detail here. Generally, the sample suspected of comprising the analyte of interest is contacted with an array produced according to the subject methods under conditions sufficient for the analyte to bind to its respective binding pair member that is present on the array. Thus, if the analyte of interest is present in the sample, it binds to the array at the site of its complementary binding member and a complex is formed on the array surface. The presence of this binding complex on the array surface is then detected, e.g. through use of a signal production system, e.g. an isotopic or fluorescent label present on the analyte, etc. The presence of the analyte in the sample is then deduced from the detection of binding complexes on the substrate surface.

Specific analyte detection applications of interest include hybridization assays in which the nucleic acid arrays of the subject invention are employed. In these assays, a sample of target nucleic acids is first prepared, where preparation may include labeling of the target nucleic acids with a label, e.g. a member of signal producing system. Following sample preparation, the sample is contacted with the array under hybridization conditions, whereby complexes are formed between target nucleic acids that are complementary to probe sequences attached to the array surface. The presence of hybridized complexes is then detected. Specific hybridization assays of interest which may be practiced using the subject arrays include: gene discovery assays, differential gene expression analysis assays; nucleic acid sequencing assays, and the like. Patents and patent applications describing methods of using arrays in various applications include: U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference.

In certain embodiments, the subject methods include a step of transmitting data from at least one of the detecting and deriving steps, as described above, to a remote location. By “remote location” is meant a location other than the location at which the array is present and hybridization occur. For example, a remote location could be another location (e.g. office, lab, etc.) in the same city, another location in a different city, another location in a different state, another location in a different country, etc. As such, when one item is indicated as being “remote” from another, what is meant is that the two items are at least in different buildings, and may be at least one mile, ten miles, or at least one hundred miles apart. “Communicating” information means transmitting the data representing that information as electrical signals over a suitable communication channel (for example, a private or public network). “Forwarding” an item refers to any means of getting that item from one location to the next, whether by physically transporting that item or otherwise (where that is possible) and includes, at least in the case of data, physically transporting a medium carrying the data or communicating the data. The data may be transmitted to the remote location for further evaluation and/or use. Any convenient telecommunications means may be employed for transmitting the data, e.g., facsimile, modem, internet, etc.

As such, in using an array made by the method of the present invention, the array will typically be exposed to a sample (for example, a fluorescently labeled analyte, e.g., protein containing sample) and the array then read. Reading of the array may be accomplished by illuminating the array and reading the location and intensity of resulting fluorescence at each feature of the array to detect any binding complexes on the surface of the array. For example, a scanner may be used for this purpose which is similar to the AGILENT MICROARRAY SCANNER scanner available from Agilent Technologies, Palo Alto, Calif. Other suitable apparatus and methods are described in U.S. patent applications: Ser. No. 09/846,125 “Reading Multi-Featured Arrays” by Dorsel et al.; and Ser. No. 09/430,214 “Interrogating Multi-Featured Arrays” by Dorsel et al. As previously mentioned, these references are incorporated herein by reference. However, arrays may be read by any other method or apparatus than the foregoing, with other reading methods including other optical techniques (for example, detecting chemiluminescent or electroluminescent labels) or electrical techniques (where each feature is provided with an electrode to detect hybridization at that feature in a manner disclosed in U.S. Pat. No. 6,221,583 and elsewhere). Results from the reading may be raw results (such as fluorescence intensity readings for each feature in one or more color channels) or may be processed results such as obtained by rejecting a reading for a feature which is below a predetermined threshold and/or forming conclusions based on the pattern read from the array (such as whether or not a particular target sequence may have been present in the sample or an organism from which a sample was obtained exhibits a particular condition). The results of the reading (processed or not) may be forwarded (such as by communication) to a remote location if desired, and received there for further use (such as further processing).

Kits

Finally, kits for use in analyte detection assays are provided. The subject kits at least include the arrays of the subject invention. The kits may further include one or more additional components necessary for carrying out an analyte detection assay, such as sample preparation reagents, buffers, labels, and the like. As such, the kits may include one or more containers such as vials or bottles, with each container containing a separate component for the assay, and reagents for carrying out an array assay such as a nucleic acid hybridization assay or the like. The kits may also include a denaturation reagent for denaturing the analyte, buffers such as hybridization buffers, wash mediums, enzyme substrates, reagents for generating a labeled target sample such as a labeled target nucleic acid sample, negative and positive controls and written instructions for using the subject array assay devices for carrying out an array based assay. The instructions may be printed on a substrate, such as paper or plastic, etc. As such, the instructions may be present in the kits as a package insert, in the labeling of the container of the kit or components thereof (i.e., associated with the packaging or sub-packaging) etc. In other embodiments, the instructions are present as an electronic storage data file present on a suitable computer readable storage medium, e.g., CD-ROM, diskette,

The following examples are offered by way of illustration and not by way of limitation.

### Experimental

A. Description of the Automated Array Manufacturing Device

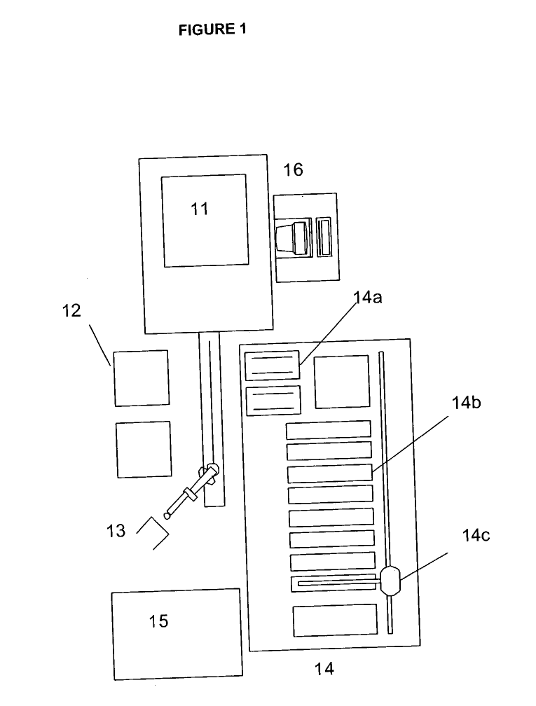

An automated device suitable for practicing the methods of the subject invention is shown schematically in FIG. 1. The device shown in FIG. 1 is an integrated and fully automated nucleic acid array production device. The system has five main components: (i) array writer/inspector 11 (e.g., a pulse-jet deposition component for depositing nucleoside monomers); (ii) substrate cassette load/unload station(s) 12; (iii) multi-axis substrate transfer robot 13; (iv) wet-chemical process station 14, which includes input/output stations 14a, process tanks 14b and robot 14c; and (v) chemical delivery system. Also shown is computer controller 16.

During use, the substrates cycle around this system in a loop between the writer/inspector and the wet-chemical processing station, where the number of cycles corresponds to the DNA oligo length being manufactured. For example, when the system makes oligos 25 to 60 bases in length, the system recirculates the substrates 25 to 60 times between the writer/inspector and the wet-chemical stations before returning the arrays to the load/unload station.

Within the wet chemical processing station (WCPS) each substrate is dipped into 2 or 3 process baths and rinsed in multiple solvent tanks before being dried and returned to the writer/inspector for another round of nucleoside monomer deposition.

The transfer robot picks up the substrates or wafers from the writer and places them in the input station of the WCPS. The processing robot on the WCPS then picks up the wafers from the nesting station and dips them in a vertical orientation into the multiple process baths based on the user defined recipe and throughput schedule. The wafers are transported individually or as pairs or several wafers at a time in chemical resistant carriers, between the process points and the input and output stations. The in-bath process times vary from 5 to 120 seconds. Once the wafers have completed the entire wet-chemistry process they are moved into the N₂ dryer. To ensure a completely dry substrate an N₂ dryer may incorporate elevated gas temperatures. The outgoing nesting station may accommodate multiple wafers, acting as a queue depending on the requirements of the recipe and scheduler. The transfer robot has the ability to pick wafers from multiple output slots on the WCPS.

B. Array Fabrication Protocol

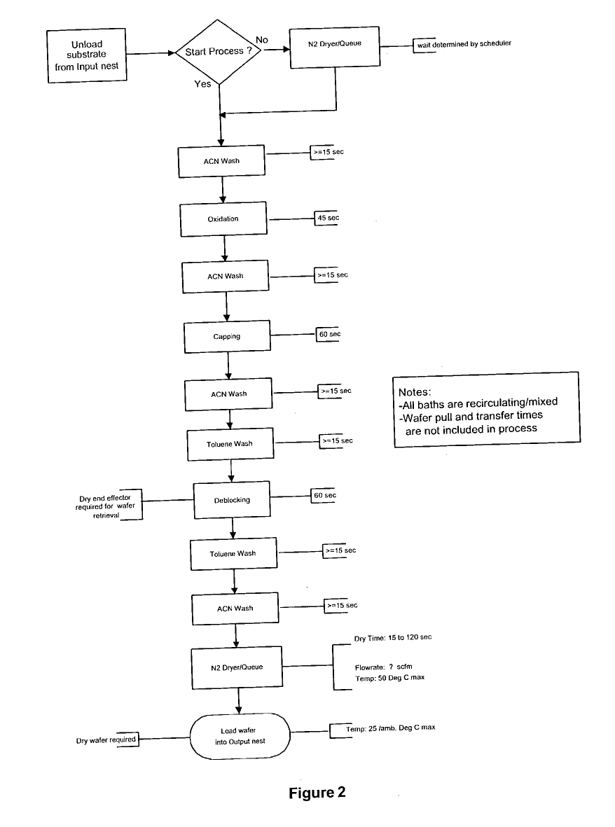

A flow chart of a representative process for using the above device in array fabrication is provided in FIG. 2. This flow chart should be used for process and scheduler modeling. The chemistries used in each process bath are provided in Table 1, below.

TABLE 1

| | |
|---|---|
| ACN Wash | acetonitrile |
| Oxidation | iodine (0.1% vol) |
| | pyridine (10% vol) |
| | water (2% vol) |
| | THF (87.9% vol) |
| ACN Wash | acetonitrile |
| Capping | CapA + CapB |
| | Cap A - acetic anhydride (10% vol) |
| | Cap A - 2,6 lutidine (10% vol) |
| | Cap A - THF (80% vol) |
| | Cap B - 1-methyl imidazole (10% vol) |
| | Cap B - THF (90% vol) |
| ACN Wash | acetonitrile |
| Toluene Wash | Toluene (100%) |
| Deblocking | Dichloroacetic acid (3% vol) |
| | Toluene (97% vol) |
| Toluene Wash | Toluene (100%) |
| ACN Wash | acetonitrile |

After exposure to any of the wash steps the wafer can be removed from the bath for an indefinite period prior to the next process step. Once the substrates are removed from the oxidation or deblock solutions, they are put into the wash tank immediately. Drying of the wafer in the N₂ dryer/queue station ensures a dry substrate before the next writing step. The use of impinging gas jets or other techniques between the wash and the deblocking step may be required.

C. Characterization Assays Performed on Arrays Made According to the Process Described in B, above.

Experimental arrays containing 33,820 features on a 3×3 inch glass wafer are prepared by in situ coupling of phosphoramidite reagents deposited by an inkjet-based apparatus using as described in B above. 3,020 of the features contain the same test element of sequence 3′-AAAAAAAAAAAAAAAAAATCTCCCA-5′ (SEQ ID NO:1) (reaction domain is in bold, detection domain is in italic) while the remaining oligonucleotides are internal, positive and negative controls. For each experiment, the deblocking agent and solvent used are varied.

The characterization of the synthesis efficiency of the array is performed by a method described in copending U.S. patent application Ser. No. 10/172,675 filed Jun. 14, 2002, the disclosure of which is herein incorporated by reference. Briefly, the experimental arrays are hybridized with an excess of a mixture of Cy3/Cy5 labeled oligonucleotides complementary to the detection domain (5′-Cy3/Cy5-TAGAGGGT-3′) and, if necessary, to other sequences present on the arrays. The hybridization buffer, temperature, duration and washing conditions were those recommended in the Agilent hybridization kit (Agilent Technologies Inc., Palo Alto, Calif.). Fluorescent detection of the hybridized, labeled target is performed on a G2565AA Agilent DNA microarray scanner (Agilent Technologies Inc., Palo Alto, Calif.) and data analysis is performed using Access, Excel, Spotfire and standard user created macros to average and display the data.

For each array synthesized under a different deblock process, the signal intensity of features of same sequence are averaged and the standard deviations calculated. Per the experiment design, the averaged signal is inversely proportional to the extent of depurination which occurred during the synthesis, thus allowing the determination of the best deblock agent/solution combination to maximize signal and minimize standard deviations.

It is evident from the above results and discussion that an important new protocol for preparing nucleic acid arrays is provided by the subject invention. Specifically, the subject methods provide for automated protocols of in situ synthesis of nucleic acid arrays with greatly reduced depurination side reactions resulting from the deblocking step, resulting in in situ production of arrays with highly uniform features. Accordingly, the subject invention represents a significant contribution to the art.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

What is claimed is:

1. A method of producing an array of at least two different nucleic acid ligands covalently bonded to a surface of a substrate, said method comprising:
(a) providing a substrate having a surface displaying functional groups;
(b) contacting blocked nucleoside monomers to at least a first location and a second location of said surface under conditions sufficient for said blocked nucleoside monomers to covalently bond to said surface in said first and second locations to produce a substrate surface displaying covalently bound blocked monomers, wherein said blocked nucleoside monomers are blocked at their 5′ end with a labile blocking group;
(c) contacting said surface displaying blocked nucleoside monomers with an oxidation solution to produce an oxidized surface;
(d) contacting said oxidized surface with a deblocking solution that comprises a deblocking agent in an organic solvent having a vapor pressure at standard temperature and pressure (STP) of less than about 13 KPa to produce a deblocked surface;
(e) removing excess deblocking solution from said deblocked surface; and
(f) reiterating steps (b) to (e) at least once to produce said array of at least two different nucleic acid ligands.

2. The method according to claim 1, wherein said labile blocking group is an acid labile blocking group.

3. The method according to claim 2, wherein said acid labile blocking group is dimethoxytrityl (DMT).

4. The method according to claim 1, wherein said surface is contacted with a capping solution prior to said deblocking step (d).

5. The method according to claim 1, wherein said blocked nucleoside monomers are contacted with said surface by pulse-jet deposition.

6. The method according to claim 1, wherein said reacted surface is sequentially contacted according to step (c) by immersing said substrate into said oxidizing solution.

7. The method according to claim 1, wherein said oxidized surface is contacted with said deblocking solution according to step (d) by immersing said substrate into said deblocking solution.

8. The method according to claim 7, wherein excess deblocking solution is removed from said deblocked surface according to step (e) by dripping.

9. The method according to claim 8, wherein said dripping lasts for a period of time ranging from about 1 s to about 60 s.

10. The method according to claim 1, wherein said organic solvent of said deblocking solution is toluene.

11. The method according to claim 1, wherein said deblocking agent-is an acid.

12. The method according to claim 11, wherein said acid is an acetic acid.

13. The method according to claim 1, wherein said method further comprises washing said oxidized surface of said substrate with said organic solvent of said deblocking solution prior to said deblocking step (d).

14. The method according to claim 1, wherein said method further comprises washing said oxidized surface of said substrate with said organic solvent of said deblocking solution after said deblocking step (d).

15. A method of producing an array of at least two different nucleic acid ligands covalently bonded to a surface of a substrate, said method comprising:
(a) providing a substrate having a surface displaying hydroxyl functional groups;
(b) contacting DMT blocked nucleoside monomers via pulse-jet deposition to at least a first location and a second location of said surface under conditions sufficient for said DMT blocked nucleoside monomers to covalently bond to said surface in said first and second locations to produce a substrate surface displaying covalently bound blocked nucleoside monomers;
(c) immersing said substrate in a volume of an oxidation solution to produce a substrate having an oxidized surface;
(d) immersing said substrate having an oxidized surface in a volume of a deblocking solution that comprises an acid in toluene to produce a substrate having a deblocked surface;
(e) removing excess deblocking solution from said substrate having a deblocked surface by dripping; and
(f) reiterating steps (b) to (e) at least once to produce said array of at least two different nucleic acid ligands.

16. The method according to claim 15, wherein said surface is contacted with a capping solution prior to said deblocking step (d).

17. The method according to claim 15, wherein said dripping lasts for a period of time ranging from about 1 s to about 60 s.

18. The method according to claim 15 wherein said acid of said deblocking solution is an acetic acid.

19. The method according to claim 15, wherein said method further comprises washing said oxidized surface of said substrate with toluene prior to said deblocking step (d).

20. The method according to claim 15, wherein said method further comprises washing said oxidized surface of said substrate with toluene after said deblocking step (d).

21. A nucleic acid array produced according to the method of claim 1.

22. A nucleic acid array produced according to the method of claim 15.

23. A method of detecting the presence of a nucleic acid analyte in a sample, said method comprising:
(a) contacting a sample suspected of comprising said nucleic acid analyte with a nucleic acid array according to claim 21;
(b) detecting any binding complexes on the surface of the said array to obtain binding complex data; and
(c) determining the presence of said nucleic acid analyte in said sample using said binding complex data.

24. The method according to claim 23, wherein said method further comprises a data transmission step in which a result from a reading of the array is transmitted from a first location to a second location.

25. A method according to claim 24, wherein said second location is a remote location.

26. A method comprising receiving data representing a result of a reading obtained by the method of claim 24.

27. A hybridization assay comprising the steps of:
(a) contacting at least one labeled target nucleic acid sample with a nucleic acid array according to claim 1 to produce a hybridization pattern; and
(b) detecting said hybridization pattern.

28. The method according to claim 27 wherein said method further comprises washing said array prior to said detecting step.

29. The method according to claim 27, wherein said method further comprises preparing said labeled target nucleic acid sample.

30. A kit for use in a hybridization assay, said kit comprising:
a nucleic acid array produced according to the method of claim 1.

31. The kit according to claim 30, wherein said kit further comprises reagents for generating a labeled target nucleic acid sample.
